(19) United States
(12) Patent Application Publication
(10) Pub. No.: US 2003/0018079 A1
(43) Pub. Date: Jan. 23, 2003

(54) Treatment

(76) Inventors: HelenE Richardson, Uppsala (SE); Thom J. Zimmerman, Louisville, KY (US); Teresa Challoner, London (GB); Per Jonsson, Uppsala (SE); Anna Gronbladh, Enskede (SE); Patrik Ohagen, Uppsala (SE); Donald Gieseker, Bucks (GB)

Correspondence Address: DINSMORE & SHOHL, LLP, 1900 CHEMED CENTER, 255 EAST FIFTH STREET, CINCINNATI, OH, 45202, US

(21) Appl. No.: 10/035,963

(22) Filed: Nov. 9, 2001

Related U.S. Application Data
(60) Provisional application No. 60/248,123, filed on Nov. 13, 2000.

(51) Int. Cl. A61K 31/535; A61K 31/557

(52) U.S. Cl. 514/573; 514/469

(57) ABSTRACT

The present invention is directed to using two or more agents in combination with capacity of reducing the intraocular pressure in a therapy with an improved efficacy to treat advanced glaucoma in such patients who suffer from detectable vision related impairments, when said agents are administered simultaneously. The combined use will also find advantage in treatment of individuals in need of a high IOP-reduction, such as those being exposed to risk factors rendering them susceptible to visual impairments.

### BACKGROUND OF THE INVENTION

Glaucoma is generally described as a group of ocular conditions, which involve progressive optic nerve damages, and the loss of visual functions. The pathogenesis of the optical nerve damage remains unclear, but it is widely accepted that a chronic elevation of the intraocular pressure (IOP) is an important factor in glaucoma damage development. The generation of ocular hypertension is associated with an impaired circulation of aqueous humor in the eye which in many cases is the result of an imbalance between the formation of aqueous humor and impaired outflow mechanisms through the trabecular meshwork and Schlemm's canal in the anterior chamber. Conventionally, glaucoma is diagnosed if two of the three criteria among elevated IOP, optical nerve head damage and visual field loss are found in the same of eye a patient. Nevertheless, it is clinically established to prescribe a therapy to individuals, which are exposed to chronic IOP elevation in order to minimize the risk that they acquire irreparable visual damages associated with diagnosed glaucoma. The most widespread IOP-reducer has been the beta-adrenergic agent timolol, which is exerting its effect by reducing the production of aqueous humor and thereby contribute to alleviate the impaired turn-over of aqueous humor of the eye. Recent clinical developments in ophthalmology in terms of glaucoma therapy have established the prostaglandin F_{2α}. derivative latanoprost (marketed as Xalatan® by Pharmacia Corp.) as a potent and useful F_{2α} intraocular pressure reducer with few side effects. Since the IOP reducing effect of prostaglandin F_{2α} derivatives including latanoprost has been attributed to their capacity of increasing the uveoscleral outflow of aqueous humor, it has been suggested to combine it with other known IOP-reducing agents exerting their effect through a different mechanism in order to obtain an additive effect. For this reason, combination therapy with beta-adrenergic agonists was early suggested, see European Patent No. 0286903 and U.S. Pat. Nos. 5,405,846 and 5,166,175. For example, P Hoyng et al in Survey Ophthalmol. 1997, 41(Suppl. 2), S93 disclose studies made on latanoprost and timolol that demonstrates an additive IOP-reducing effect in patients suffering from an elevated IOP and having an insufficient response to timolol alone. There are several studies directed to investigate the IOP reducing effects from adjunctive therapy of the beta-adrenergic agonist timolol and latanoprost, which suggest that the combination results in a more pronounced hypotensive effect than can be achieved from any of the two drugs alone, see N Pfieffer in IOVS 2000, 41(4), S754; B Sjöquist et al in IOVS 2000, 41(4), S572; L I Larsson in IOVS 2000, 41(4), S280; P Hyong et al in Drugs 2000, 59(3), 411-434; W C Stewart et al in J Ocul Pharmacol Ther, 2000, 16(3), 251-259; K Iishi et al in Jpn J Ophthalmol, 2000, 44(3), 227-234; P T Hung et al in Am J Ophthalmol, 1999, 128(6), 692-696; P G Watson in Drugs Today, 1999, 35(6), 449-459; C Linden et al in Drugs Aging, 1999, 14(5), 387-398; L Martin in Acta Ophthalmol Scand, 1999, 77(3), 336-339; T W Heijkal et al in Seminars in Ophthalmology, 1998, 14(3), 114-123; M Diestelhorst et al in Graefe's Arch Clin Exp Ophthalmol, 1998, 236(8), 577-581 and A Alm et al in British J Ophthalmol, 1995, 79(1), 12-6. Furthermore, there are several non-prostaglandin containing fixed combinations available for the treatment of glaucoma based on a beta-adrenergic antagonist and a complementary agent with ocular hypotensive effect. Normoglaucon® contains 0.1% metipranolol and 2% pilocarpine. TP-2® or Timpilo-2® contains 0.5% timolol and 2% pilocarpine. Cosopt® contains 0.5% timolol and 2% dorzolamide.

Given that the course of development of glaucoma is unpredictable with a pathogenesis largely varying among individuals, frequently with unnoticeable symptoms and signs, certain patients may have reached an advanced stage of the disease with visual field loss as a result of optical nerve damage, even before they are examined by medical expertise. For this type of patients, it is necessary to institute a radical IOP-reducing treatment. However, conventional IOP-reducers frequently are insufficient to reach suitable results and surgical intervention may be necessary to restore the turn-over of aqueous humor by improving its outflow. Although treatments with combination of IOP-reducing agents which affect the IOP-reduction according to different mechanisms have been suggested to generate additive effects beyond each individual agent, there are so far no indications that any combination therapy would have an especial efficacy for patients suffering from advanced glaucoma. It would therefor be desirable to provide for a therapeutic treatment that was especially efficient in reaching such patients who are suffering from these advanced stages of glaucoma who are at serious risk to acquire further loss of vision to an extent that would compromise their quality of life.

### DESCRIPTION OF INVENTION

It is an object of the present invention to provide for a therapy according to which particular high-risk glaucoma patients can be treated with greater efficacy.

It is another object of the present invention to provide for a therapy for patients with a particular risk factor of acquiring advanced glaucoma can be treated with higher efficacy.

It is a particular object of the present invention to employ a combination of IOP-reducing agents for simultaneous administration and thereby obtain an improved IOP-reducing efficacy in severe glaucoma patients and individuals having an especial need of a high IOP reduction.

The present invention resides in the finding that a therapy of two or more agents with capacity of reducing the intraocular pressure has an improved efficacy to treat advanced glaucoma in such patients who suffer from detectable vision related impairments, when said agents are administered simultaneously. In the inventive context, simultaneous administration means that the agents are delivered to the eye substantially at the same time, for example subsequently immediately after each other, or that they are co-administered as a mixture. Dependent on the characteristics of the agents they can be pre-mixed in a ready-made solution, or for stability reasons separately stored and mixed, just prior to the administration. There are many devices available to skilled practitioners to prepare a solution in-situ and these are not described in any detail herein as they not are a part of the present invention. It is preferred that the combination is a mixture of agents that can be applied to the surface of the eye in the form of a topical ophthalmic preparation delivered in drop form or delivered in the form of a directed stream from a pressurized ophthalmic dispenser.

It has been surprisingly found that the IOP reducing capacity arrived from a combination treatment in such patients significantly exceeds IOP reduction in patients exposed to an IOP increase, who thereby are at risk of obtaining visual damages, but not yet having acquired such advanced stages of the ailment. The inventive method will be particularly useful for the mentioned patients and also for individuals in particular need of a high reduction of IOP due to the exposure of certain risk factors which can be considered to aggravate or accelerate the visual complications arriving from exposure to ocular hypertension. Such individuals include those who belong to family with a history of glaucoma cases and individuals suffering from conditions which may trigger ischemic complications in the region of the optical nerve head. The skilled practitioner will be able to sort out individuals who would be extra susceptible to acquire damages from elevated IOP and thereby will be elected to undergo a combination therapy.

In the context of the present invention advanced glaucoma or severe glaucoma shall be defined as a condition where an individual has acquired an optical nerve damage, i.e. abnormalities of the optical nerve head and defects of the visual field. Both these damages can be detected by standard methods available to ophthalmologists. The presence of an optical nerve damage can be objectively measured for example by laser scanning tomography to measure the nerve fiber thickness, see L M Zangwill et al. Optometry and Vision Science, 1999, 76(8), pp. 526-36 or the similar methods to objectively estimate the loss of tissue. Visual field loss can be measured by conventional methods employed by ophthalmologists.

In further context of the present invention, a combination of IOP reducers is defined as at least two different agents with IOP reducing capacity acting according to different mechanisms in their to provide the reduction when they are concomitantly administered. For example, such differences in mechanistic onset of the IOP-reduction could include stimulation (affinity to) of different receptors in the eye, however, not necessary located at different sites of the eye. Accordingly, different prostaglandin derivatives with different prostaglandin receptor profiles can be used, such as a prostaglandin derivative predominantly exerting its IOP-receptor effect through the FP receptor could be combined with one or several prostaglandins exerting is IOP-reducing effect less selectively by a pronounced affinity to other of eight major prostaglandin receptors.

Preferably, a combination of IOP-reducers having different physiological actions is used in the present invention. A suitable combination would be one agent increasing the outflow of aqueous humor and one agent reducing its formation of aqueous humor. A typical combination of an IOP reducing effective amount of a prostaglandin derivative together with at least one IOP reducing agent exerting its activity through other receptors than prostaglandin receptors. Particularly useful are prostaglandins or prostaglandin derivatives capable of reducing IOP by increasing the uveoscleral outflow in combination with one or several IOP-reducing agents having another physiological action. Such prostaglandins are found among prostaglandin F_{2α} (PGF_{2α}) analogues and derivatives such as those discussed in U.S. Pat. No. 4,599,353. Preferably, the prostaglandin F_{2α} derivatives have the carboxyl group in the alpha-chain substituted with a lower alkyl ester, such as isopropyl ester, to improve corneal penetration. Alternatively, said carboxyl group can be substituted with alcohol or ether or the similar for rendering the compound more lipophilic. Especially useful such PGF_{2α}. derivatives have ring-formed substituent in the terminal of the omega-chain of the prostaglandin F_{2α} structure, such as 13,14-dihydro-17-phenyl-18,19,20-trinor-prostglandin F_{2α}-isopropyl ester (latanoprost), 16-(meta-trifluromethyl)-phenoxy-17,18,19,20-tetranor-prostglandin F_{2α}-isopropyl ester (travaprost) and similar compounds referred to in WO 90/02553. Ring-formed substituent is defined as an aryl group, an arylalkyl group, a heterocyclic aromatic group or a cycloalkyl group which optionally is substituted. Also useful, however less potent than the aforementioned compounds, is the PGF_{2α}-metabolite analogue isopropyl unoprostone. Numerous other prostaglandin derivatives are described in the literature as ocular hypotensive agents or anti-glaucoma agents under denominations deviating from prostaglandin nomenclature, such as hypotensive lipids and the similar. Obviously, such compounds also will be a part of the present invention.

An IOP-reducing prostaglandin according what is stated above preferably is combined with at least one IOP reducing agent selected among cholinergic agonists (such as pilocarpine), beta-adrenegic antagonists (such as timolol), carbonic anhydrase inhibitors (such as acetazoloamide or dorzolamide) or beta-adrenergic agonists (such as dipivefrine). More suitably, said prostaglandin is combined with one or several IOP-reducing agent capable of affecting the formation of the aqueous humor, such as a carbonic anhydrase inhibitor or a beta-adrenergic antagonist (beta-blocker). Especially preferred is a combination of a prostaglandin and a beta-adrenergic antagonist in the form of an ophthalmically acceptable composition for topical administration to the eye. Suitably the prostaglandin is a prostaglandin F_{2α} derivative with capacity of increasing the uveoscleral outflow, such as latanoprost, travaprost or isopropyl unoprostone. The beta-adrenergic antagonist is selected among conventional such agents including, but not limited to, acebutolol, alprenolol, atenolol, betaxolol, bisoprolol, carteolol, celiprolol, esmolol, labetalol, levobunolol, metipranolol, metoprolol, nadolol, oxprenolol, penbutolol, pindolol, propranolol, sotalol, and timolol. Especially preferable beta-adrenergic antagonist are timolol maleate, betaxolol hydrochloride, levobunolol hydrochloride and metipranolol.

The inventive therapy is conducted with regular doses of the combination, such as in the form of eye drops each having a volume of about 30 μl. Typically such a dose comprises about 0.1 to 1000 μg, preferably 0.1 to 50 μg of prostaglandin derivative and beta-adrenergic agents in the range of about 0.01 μg to 1000 μg, preferably from about 5 μg to 500 μg.

An especially preferred combination is a topical ophthalmic composition of the PGF_{2α} derivative latanoprost and the beta-blocker timolol. The composition further comprises conventional additives rendering it suitable for topical ophthalmic administration, such as preservatives and solubilizers. Typically, such a composition comprises from about 0.001 to 0.01% (w/v) of latanoprost and from about 0.1 to 2% (w/v) of timolol.

A greatly preferred composition to included in the combination comprises 0.5% (5 mg/ml) timolol and 0.005% (50 μg/ml) latanoprost together with one or several buffering agents, a preservative or solubilizer, a tonicity agent and one or several pH adjustment agents. A specific example of composition useful in the present invention contains:

| Name of Ingredient | Concentration (mg/ml) | | Function |
|---|---|---|---|
| Latanoprost | 50 | μg | Active ingredient |
| Timolol maleate | 6.83 | mg | Active ingredient |
| Benzalkonium chloride | 200 | μg | Preservative/solubilizer |
| Disodium phosphate | 2.89 | mg | Buffering agent |
| anhydrous | | | |
| Sodium dihydrogen | 6.39 | mg | Buffering agent |
| phosphate | | | |
| monohydrate | | | |
| Sodium chloride | 4.10 | mg | Tonicity agent |
| 10% solution | q.s. to pH 6.0 | | pH adjustment |
| Hydrochloric acid | if required | | |
| 10% solution Sodium | q.s. to pH 6.0 | | pH adjustment |
| Hydroxide | if required | | |
| Water for injection | ad 1.00 | ml | Solvent |

The composition will be packaged as a sterile eye drops product in 5 ml bottles suitable for administering 30 μl drop dosages to the surface of the eye.

In the following experimental section, it has been demonstrated that a combination therapy as exemplified with the combination of latanoprost and timolol has an unexpected efficacy for patients suffering from severe glaucoma.

### EXEMPLIFYING PART OF THE DESCRIPTION

A sub-population of 76 individuals in a population of total 854 patients enrolled into two different studies of German patients (004) and US patients (005) were identified at baseline as having some degree of abnormality to the optic nerve head together with a glaucomatous visual field defect and were treated with a fixed combination (FC) of latanoprost and timolol. Both studies were based on a randomized double-masked parallel group design. In both studies, a fixed combination (FC) of latanoprost and timolol was administered to a group of patients with optic nerve head damage and visual field loss (i.e. glaucomatous field defects) and to groups of patients without any such detected damages, but with an elevation of IOP. Patient demography and baseline characteristics in patients with and without optic nerve head damages and glaucomatous field defects are shown in Table 2.1.

The patients is the studies received one drop in the morning of a fixed combination of latanoprost (50 μg/ml) and timolol (5 mg/ml) during the study duration of 26 weeks. The exact composition of fixed combination is disclosed in Table 1. At baseline, IOP assessments were made at 08:00, 10:00, and 16:00. Measurements at the same time-points were subsequently made at scheduled clinic visits at Week 2, Week 13, and Week 26. Additionally, an 08:00 measurement was also obtained at Week 6. The patients have an approximately 5 mm Hg decrease in IOP from a timolol run-in period.

Comparisons of Tables 2.2 and 2.4 related to study 004 and comparisons of Tables 2.3 and 2.5 related to study 005 demonstrates that the mean reduction in IOP (i.e. mean change from baseline) is significantly higher for patients suffering from both abnormalities of the optic nerve head and visual field defects when compared to patients having an elevated IOP but otherwise free from the mentioned complications. From these results, it is evident that the Fixed Combination (FC) of latanoprost and timolol shows an unexpected efficacy in the mentioned patient group suffering severe or advance glaucoma.

TABLE 1
Fixed combination of eye drops latanoprost
50 μg/ml and timolol 5 mg/ml, pH = 6.0

| | |
|---|---|
| Name of Ingredients | Amount per ml |

| | | |
|---|---|---|
| Latanoprost | 50 | μg |
| Timolol maleate (equivalent to | 6.83 | mg |
| 5 mg timolol) | | |
| Polysorbate 80 | 0.05 | mg |
| Benzalkonium chloride | 0.10 | mg |
| Disodium phosphate anhydrous | 2.89 | mg |
| Sodium dihydrogen phosphate | 6.39 | mg |
| monohydrate | | |
| Sodium chloride | 4.10 | mg |
| Water for injection | ad 1.00 | ml |

TABLE 2.1
Patient demography and baseline characteristics in patients with
and without optic nerve head abnormalities and glaucomatous visual
field defects (studies 004 and 005)

| | Patients with ONH | Patients |
|---|---|---|
| Variables | damage | without ONH damages |
| Number of patients | 76 | 202 |
| Gender, n (%) | | |
| Male | 39 (51%) | 95 (47%) |
| Female | 37 (49%) | 107 (53%) |
| Age (years), | | |
| Mean (SD) | 64 (12) | 62 (13) |
| Min-Max | 24-83 | 18-86 |
| Age class n (%) | | |
| <60 years | 25 (33%) | 78 (39%) |
| 60-70 years | 27 (36%) | 67 (33%) |
| ≧70 years | 24 (32%) | 57 (28%) |
| Ethnic origin, n | | |
| (%) | 63 (83%) | 166 (82%) |
| Caucasian | 10 (13%) | 28 (14%) |
| Black | 1 (1%) | 0 |
| Asian | 0 | 1 (<1%) |
| Oriental | 1 (1%) | 6 (3%) |
| Hispanic | 0 | 0 |
| American Indian | 1 (1%) | 1 (<1%) |
| Other | | |
| Diagnosis of study | | |
| eye(s), n (%) | | |
| POAG | 66 (87%) | 134 (66%) |
| Exfoliation | 2 (3%) | 2 (1%) |
| Glaucoma | 2 (3%) | 5 (2%) |
| Pigmentary | 6 (8%) | 57 (28%) |
| Glaucoma | 0 | 4 (2%) |
| Ocular | | |
| Hypertension | | |
| Mixed diagnosis | | |
| Eye color study | | |
| eye(s), n* (%) | | |
| Homogeneously | 22 (29%) | 59 (29%) |
| blue, gray or green | | |
| Homogeneously | 21 (28%) | 69 (34%) |
| brown | | |
| Blue-brown/gray- | 24 (32%) | 57 (28%) |
| brown | | |
| Green-brown | 8 (11%) | 12 (6%) |
| Yellow-brown | 1 (1%) | 5 (2%) |
| Duration of | | |
| therapy, n* (%) | | |
| <6 months | 11 (13%) | 30 (15%) |
| 6-36 months | 9 (12%) | 53 (26%) |
| 36-100 months | 31 (41%) | 59 (29%) |
| >100 months | 25 (33%) | 60 (30%) |
| Glaucoma meds at | | |
| entry, n (%) | | |
| >one | 41 (54%) | 90 (45%) |
| one or none | 35 (46%) | 112 (55%) |
| Family history of | 21 (28%) | 62 (31%) |
| OH/glaucoma, n* | | |
| (%) | | |

TABLE 2.2
Mean change in IOP (mmHg) from baseline and differences between
treatments at each time point during the study treatment period, study 094
(patients with abnormalities of ONH and visual field defects)

FC
42 patients

| | | | Mean baseline |
|---|---|---|---|
| | | IOP | change in IOP |
| Time | Visit | (mmHg) | (mmHg) |
| | Baseline | 22.5 | |
| 08:00 | Week 2 | 18.8 | −3.7 |
| | Week 6 | 18.8 | −3.7 |
| | Week 13 | 19.2 | −3.3 |
| | Week 26 | 19.1 | −3.4 |
| 10:00 | Baseline | 22.2 | |
| | Week 2 | 18.4 | −3.9 |
| | Week 13 | 20.0 | −2.2 |
| | Week 26 | 18.7 | −3.5 |
| 16:00 | Baseline | 21.8 | |
| | Week 2 | 18.4 | −3.4 |
| | Week 13 | 18.4 | −3.4 |
| | Week 26 | 18.5 | −3.3 |

TABLE 2.3
Mean change in IOP (mmHg) from baseline and differences between
treatments at each time point during the study treatment period, study 005
(patients with abnormalities of ONH and visual field defects)

FC
34 patients

| | | | Mean baseline |
|---|---|---|---|
| | | IOP | change in IOP |
| Time | Visit | mmHg | (mmHg) |
| 08:00 | Baseline | 24.6 | |
| | Week 2 | 20.0 | −4.6 |
| | Week 6 | 19.9 | −4.7 |
| | Week 13 | 20.1 | −4.4 |
| | Week 26 | 20.7 | −3.9 |
| 10:00 | Baseline | 22.8 | |
| | Week 2 | 20.0 | −2.8 |
| | Week 13 | 19.5 | −3.3 |
| | Week 26 | 19.9 | −2.9 |
| 16:00 | Baseline | 22.9 | |
| | Week 2 | 19.1 | −3.8 |
| | Week 13 | 18.2 | −4.8 |
| | Week 26 | 19.6 | −3.3 |

TABLE 2.4
Mean change in IOP (mmHg) from baseline and differences between
treatments at each time point during the study treatment period, study 004
(patients without abnormalities of ONH and visual field defects)

FC
98 patients

| | | | Mean baseline |
|---|---|---|---|
| | | IOP | change in IOP |
| Time | Visit | mmHg | mmHg |
| 08:00 | Baseline | 22.2 | |
| | Week 2 | 19.8 | −2.4 |
| | Week 6 | 19.4 | −2.9 |
| | Week 13 | 19.5 | −2.7 |
| | Week 26 | 19.5 | −2.7 |
| 10:00 | Baseline | 21.4 | |
| | Week 2 | 19.0 | −2.4 |
| | Week 13 | 18.9 | −2.5 |
| | Week 26 | 19.3 | −2.1 |
| 16:00 | Baseline | 20.6 | |
| | Week 2 | 18.3 | −2.3 |
| | Week 13 | 18.2 | −2.4 |
| | Week 26 | 18.3 | −2.3 |

TABLE 2.5
Mean change in IOP (mmHg) from baseline and differences between
treatments at each time point during the study treatment period, study 005
(patients without abnormalities of ONH and visual field defects)

FC
104 patients

| | | | Mean change in |
|---|---|---|---|
| | | IOP | IOP from baseline |
| Time | Visit | mmHg | mmHg |
| 08:00 | Baseline | 24.1 | |
| | Week 2 | 20.9 | −3.2 |
| | Week 6 | 20.5 | −3.6 |
| | Week 13 | 20.7 | −3.4 |
| | Week 26 | 20.6 | −3.5 |
| 10:00 | Baseline | 22.8 | |
| | Week 2 | 19.9 | −3.0 |
| | Week 13 | 19.7 | −3.2 |
| | Week 26 | 20.0 | −2.8 |
| 16:00 | Baseline | 22.0 | |
| | Week 2 | 18.8 | −3.2 |
| | Week 13 | 18.7 | −3.2 |
| | Week 26 | 19.0 | −2.8 |

1. A method of treating patients suffering from severe glaucoma characterized by simultaneously administering a combination of IOP reducing agents to the eye.

2. A method according to claim 1, wherein said combination is administered to the surface of the eye.

3. A method according to claim 2, wherein said combination is a topical ophthalmic composition comprising a mixture of IOP-reducing agents.

4. A method according to claim 1, wherein said patients suffer from optical nerve head damage and visual field defects.

5. A method according to claim 1, wherein in improved efficacy in IOP reduction is obtained in severe glaucoma patients when compared to patients suffering from an elevated IOP, but being free from abnormalities in the optical nerve head and visual field loss.

6. A method according to claim 1, wherein said combination comprises an effective amount of an IOP-reducing agent capable of increasing the uveoscleral outflow of aqueous humor.

7. A method according to claim 1, wherein said combination comprises an effective amount of an IOP reducing prostaglandin or a prostaglandin derivative.

8. A method according to claim 7, wherein said combination comprises an IOP reducing amount of a prostaglandin F_{2α} derivative.

9. A method according to claim 8, wherein said prostaglandin F_{2α} derivative has an omega chain carrying a ring substituent in a terminal position, selected among optionally substituted phenyl, cycloalkyl or aromatic heterocyclic groups.

10. A method according to claim 9, wherein said prostaglandin F_{2α} is latanoprost or travaprost.

11. A method according to claim 8, wherein said prostaglandin F_{2α} derivative is isopropyl unoprostone.

12. A method according to claim 1, wherein said combination comprises an effective amount of an IOP-reducing agent capable of reducing the formation of aqueous humor.

13. A method according to claim 12, wherein said combination further comprises an effective amount of an IOP-reducing agent capable of increasing the uveoscleral outflow of aqueous humor.

14. A method according to claim 12, wherein said IOP-reducing agent is selected among beta-adrenergic agonists and carbonic anhydrase inhibitors.

15. A method according to claim 14, wherein said combination comprises a prostaglandin F_{2α} derivative and a beta-adrenergic agonist.

16. A method according to claim 15, wherein said combination comprises a prostaglandin F_{2α} derivative having an omega chain carrying a ring substituent in a terminal position, selected among optionally substituted phenyl, cycloalkyl or aromatic heterocyclic groups.

17. A method according to claim 16, wherein said combination comprises latanoprost and timolol.

18. A method according to claim 17, wherein said combination is a mixture of latanoprost and timolol in a topical ophthalmic composition.

19. A method of treating individuals in need of a high IOP-reduction characterized by simultaneously administering a combination of IOP reducing agents to eye.

20. A method according to claim 19, wherein said individuals have a hereditary disposition for glaucoma.

21. A method according to claim 19, wherein said individuals suffer from complications which may trigger ischemic conditions in the region of the optical nerve head.

22. A method according to claim 19, wherein said individuals suffer ocular hypertension without detected damages of the optical nerve head or a loss of the visual field.

23. A method according to claim 19, wherein said combination is administered to the surface of the eye.

24. A method according to claim 21, wherein said combination is a topical ophthalmic composition comprising a mixture of IOP-reducing agents.

25. A method according to claim 19, wherein said combination comprises an effective amount of an IOP-reducing agent capable of increasing the uveoscleral outflow of aqueous humor.

26. A method according to claim 19, wherein said combination comprises an effective amount of an IOP reducing prostaglandin or a prostaglandin derivative.

27. A method according to claim 26, wherein said combination comprises an IOP reducing amount of a prostaglandin F_{2α} derivative.

28. A method according to claim 27, wherein said prostaglandin F_{2α} derivative has an omega chain carrying a ring substituent in a terminal position, selected among optionally substituted phenyl, cycloalkyl or aromatic heterocyclic groups.

29. A method according to claim 28, wherein said prostaglandin F_{2α} is latanoprost or travaprost.

30. A method according to claim 29, wherein said prostaglandin F_{2α} derivative is isopropyl unoprostone.

31. A method according to claim 19, wherein said combination comprises an effective amount of an IOP-reducing agent capable of reducing the formation of aqueous humor.

32. A method according to claim 31, wherein said combination further comprises an effective amount of an IOP-reducing agent capable of increasing the uveoscleral outflow of aqueous humor.

33. A method according to claim 31, wherein said IOP-reducing agent is selected among beta-adrenergic agonists and carbonic anhydrase inhibitors.

34. A method according to claim 33, wherein said combination comprises a prostaglandin F_{2α} derivative and a beta-adrenergic agonist.

35. A method according to claim 34, wherein said combination comprises a prostaglandin F_{2α} derivative having an omega chain carrying a ring substituent in a terminal position, selected among optionally substituted phenyl, cycloalkyl or aromatic heterocyclic groups.

36. A method according to claim 35, wherein said combination comprises latanoprost and timolol.

37. A method according to claim 36, wherein said combination is a mixture of latanoprost and timolol in a topical ophthalmic composition.

38. The use of a combination of IOP-reducing agents for the preparation of a composition with improved efficacy in severe glaucoma patients.

39. The use according to claim 38 for the preparation of a composition for simultaneously administering the IOP reducing agents to the eye.

40. The use according to claim 39 for the preparation of a composition for administration to the surface of the eye.

41. The use according to claim 40, wherein said composition comprises a mixture of IOP-reducing agents.

42. The use according to any of claims 38 to 41, wherein said glaucoma patients suffer from optical nerve head damages and visual field defects.

43. The use according to any of claims 38 to 42, wherein said composition improves the efficacy in IOP reduction in severe glaucoma patients when compared to patients suffering from an elevated IOP, but being free from abnormalities in the optical nerve head and visual field loss.

44. The use according any of claims 38 to 43, wherein said combination comprises an effective amount of an IOP reducing agent capable of increasing the uveoscleral outflow of aqueous humor.

45. The use according to any of claims 38 to 44, wherein said combination comprises an effective amount of an IOP reducing prostaglandin or a prostaglandin derivative.

46. The use according to claim 45, wherein said combination comprises an IOP reducing amount of a prostaglandin F_{2α} derivative.

47. The use according to claim 46, wherein said prostaglandin F_{2α} derivative has an omega chain carrying a ring substituent in a terminal position, selected among optionally substituted phenyl, cycloalkyl or aromatic heterocyclic groups.

48. The use according to claim 47, wherein said prostaglandin F_{2α} is latanoprost or travaprost.

49. The use according to claim 48, wherein said prostaglandin F_{2α} derivative is isopropyl unoprostone.

50. The use according to claim 38, wherein said combination comprises an effective amount of an IOP-reducing agent capable of reducing the formation of aqueous humor.

51. The use according to claim 50, wherein said combination further comprises an effective amount of an IOP-reducing agent capable of increasing the uveoscleral outflow of aqueous humor.

52. The use according to claim 50, wherein said IOP-reducing agent is selected among beta-adrenergic agonists and carbonic anhydrase inhibitors.

53. The use according to claim 51, wherein said combination comprises a prostaglandin F_{2α} derivative and a beta-adrenergic agonist.

54. The use according to claim 53, wherein said combination comprises a prostaglandin F_{2α} derivative having an omega chain carrying a ring substituent in a terminal position, selected among optionally substituted phenyl, cycloalkyl or aromatic heterocyclic groups.

55. The use according to claim 54, wherein said combination comprises latanoprost and timolol.

56. The use according to claim 55, wherein said combination is a mixture of latanoprost and timolol in a topical ophthalmic composition.

57. The use of a combination of IOP reducing agents in the preparation of composition for simultaneous treatment with said agents of individuals in need of a high IOP reduction

58. The use according to claim 57, wherein said individuals have a hereditary disposition for glaucoma.

59. The use according to claim 57, wherein said individuals suffer from complications which may trigger ischemic conditions in the region of the optical nerve head.

60. The use according to claim 57, wherein said individuals suffer from ocular hypertension without detected damages of the optical nerve head or a loss of the visual field.

61. The use according to claim 57, wherein said combination is administered to the surface of the eye.

62. The use according to claim 61, wherein said combination is a topical ophthalmic composition comprising a mixture of IOP-reducing agents.

63. The use according to claim 57, wherein said combination comprises an effective amount of an IOP-reducing agent capable of increasing the uveoscleral outflow of aqueous humor.

64. A method according to claim 57, wherein said combination comprises an effective amount of an IOP reducing prostaglandin or a prostaglandin derivative.

65. A method according to claim 64, wherein said combination comprises an IOP reducing amount of a prostaglandin F_{2α} derivative.

66. A method according to claim 65, wherein said prostaglandin F_{2α} derivative has an omega chain carrying a ring substituent in a terminal position, selected among optionally substituted phenyl, cycloalkyl or aromatic heterocyclic groups.

67. A method according to claim 66, wherein said prostaglandin F_{2α} is latanoprost or travaprost.

68. A method according to claim 65, wherein said prostaglandin F_{2α} derivative is isopropyl unoprostone.

69. A method according to claim 57, wherein said combination comprises an effective amount of an IOP-reducing agent capable of reducing the formation of aqueous humor.

70. A method according to claim 69, wherein said combination further comprises an effective amount of an IOP-reducing agent capable of increasing the uveoscleral outflow of aqueous humor.

71. A method according to claim 69, wherein said IOP-reducing agent is selected among beta-adrenergic agonists and carbonic anhydrase inhibitors.

72. A method according to claim 71, wherein said combination comprises a prostaglandin F_{2α} derivative and a beta-adrenergic agonist.

73. A method according to claim 72, wherein said combination comprises a prostaglandin F_{2α} derivative having an omega chain carrying a ring substituent in a terminal position, selected among optionally substituted phenyl, cycloalkyl or aromatic heterocyclic groups.

74. A method according to claim 73, wherein said combination comprises latanoprost and timolol.

75. A method according to claim 74, wherein said combination is a mixture of latanoprost and timolol in a topical ophthalmic composition.
